(19) United States
(12) Patent Application Publication
(10) Pub. No.: US 2012/0172980 A1
(43) Pub. Date: Jul. 5, 2012

(54) KITS WITH PROSTHETIC VALVES FORMED WITH ISOTROPIC FILTER SCREEN LEAFLETS AND METHODS THEREOF

(75) Inventor: Jeffrey P. DuMontelle, Irvine, CA (US)

(73) Assignee: CURIA, INC., Irvine, CA (US)

(21) Appl. No.: 13/337,049

(22) Filed: Dec. 23, 2011

Related U.S. Application Data
(60) Provisional application No. 61/460,592, filed on Jan. 5, 2011.

(51) Int. Cl. A61F 2/24; B65D 71/00

(52) U.S. Cl. 623/2.12; 206/570

(57) ABSTRACT

In one embodiment of the invention, a catheter delivery system or kit is disclosed. The kit contains a prosthetic check valve with leaflets cut from filtration screen material with uniform pore size having openings with a dimension inclusively between fifteen and sixty microns. The screen material is made from biocompatible material, such as polyester or polypropylene. One or more outer edges of the leaflet are fused or sealed to prevent fraying of the material and to form a more non-thrombogenic surface. Prosthetic check valves assembled with the leaflets can be collapsed to a diameter of less than or equal to twenty-nine french (29 f), sterilized, and stored in a collapsed state. The compressed or collapsed valve may be sterilized and included as part of a sterile catheter delivery system or kit, ready for minimally invasive implantation without rinsing prior to use.

### CROSS-REFERENCE TO RELATED APPLICATIONS

The patent application claims the benefit of U.S. Provisional Patent Application No. 61/460,592 entitled FLEXIBLE LEAFLET AND FLEXIBLE LEAFLET VALVE filed on Jan. 5, 2011 by inventor Jeffrey Paul DuMontelle which is incorporated herein by reference.

### FIELD

The embodiments of the invention relate generally to prosthetic valves.

### BACKGROUND

There are four valves in the heart that serve to direct blood flow through the two sides of the heart. On the left (systemic) side of the heart are: (1) the mitral valve, located between the left atrium and the left ventricle, and (2) the aortic valve, located between the left ventricle and the aorta. These two valves direct oxygenated blood from the lungs through the left side of the heart and into the aorta for distribution to the body. On the right (pulmonary) side of the heart are: (1) the tricuspid valve, located between the right atrium and the right ventricle, and (2) the pulmonary valve, located between the right ventricle and the pulmonary artery. These two valves direct de-oxygenated blood from the body through the right side of the heart and into the pulmonary artery for distribution to the lungs, where the blood becomes re-oxygenated in order to begin the circuit anew.

All four of these heart valves are passive structures in that they do not themselves expend any energy and do not perform any active contractile function. They consist of moveable features, generally described as leaflets or cusps, that open and close in response to differential pressures on either side of the valve. The mitral and tricuspid valves are typically referred to as atrioventricular valves because they are situated between an atrium and ventricle on each side of the heart. The mitral valve has two leaflets and the tricuspid valve has three. The aortic and pulmonary valves are typically referred to as semilunar valves because of the unique appearance of their leaflets, which are shaped somewhat like a half-moon and are typically described as cusps. The aortic and pulmonary valves each have three cusps.

There are other valves in the body such as those in the peripheral vasculature. These valves are usually single leaflet structures. These valves act as gates to prevent the blood from flowing backward. For example as a leg muscle contracts, the venous vessels are compressed and the blood is pushed through a valve, as the muscle relaxes, the blood is not allowed to flow backward as the valve closes. The one-way venous valve ensures that the blood flows in one direction back towards the heart.

Human heart valve leaflets are generally formed of an endothelial layer of tissue that encloses a core of collagenous bundles. The endothelial layer has relatively few elastic fibers. The endothelium covering the surface of a leaflet typically has fine folds or corrugations that extended along this surface in a proximo-distal direction. The collagenous fibers are condensed in the center of the leaflet but loosely disposed beneath the surface endothelium. The central fibers are arranged into wavy bundles that run parallel with the base-to-apex axis of the leaflet near its atrial aspect and they are typically obliquely or irregularly disposed towards the ventricular aspect of the leaflet. The elastic fibers are typically found only in the subendothelial zone of the leaflet. The leaflet core is thicker at the peripheral basal margin of a leaflet and tapering towards its central free margin. The endothelial layers have minimal stretch but the internal collagenous structure acts as flexible structure that compensates for the differences in lengths as the valve opens and closes. This multilayer type configuration allows the leaflet to readily flex back and forth from a concave to a convex condition.

Valves may exhibit abnormal anatomy and function as a result of congenital or acquired valve disease. Congenital valve abnormalities may be well-tolerated for many years only to develop a life-threatening problem in an elderly patient, or may be so severe that emergency surgery is required in-utero or within the first few hours of life. Acquired valve disease may result from causes such as rheumatic fever, degenerative disorders of the valve tissue, bacterial or fungal infections, and trauma.

Since valves are passive structures that simply open and close in response to differential pressures on either side of the particular valve, failure modes that can develop with valves can be classified into two categories: (1) stenosis, in which a valve does not open properly, and (2) insufficiency (also called regurgitation), in which a valve does not close properly. Stenosis and insufficiency may occur at the same time in the same valve or in different valves.

Both of these abnormalities increase the workload placed on the heart as well as other organs of the body such as the liver and kidneys. In particular, the severity of this increased stress on the heart, and the heart's ability to adapt to it, determine whether the abnormal valve will have to be repaired or surgically removed and replaced. The functions of the valve may be replaced by implantation of an additional valve within the diseased valve.

Development of a prosthetic valve that can approach the overall performance of a native valve requires that such a valve be biocompatible, durable, non-thrombogenic, and exhibit advantageous hemodynamic performance.

Valve replacement surgery or valve function replacement is described and illustrated in numerous books and articles, and a number of options, including artificial mechanical valves and artificial bioprosthetic (tissue) valves, are currently available. However, the currently-available options cannot completely duplicate the advantages of native (natural) valves. Some of the available mechanical valves tend to be very durable, but are problematic in that they are thrombogenic, exhibit relatively poor hemodynamic properties and generally cause significant damage to the patient's blood cells when they are performing their function (i.e. opening and closing in the blood flow). All of these characteristics usually require lifelong anticoagulation therapy for the patient.

Some of the available bioprosthetic tissue valves may have relatively low thrombogenicity, but lack the durability of mechanical valves. This lack of durability is generally attributed to the tissue material used in the valves, where the material properties may not be well understood and therefore used improperly in the design of the valve. The material itself may be attacked by the patient's normal body defenses, resulting in calcification of the leaflets, and ultimately, failure of the valve.

Mechanical valves such as the caged ball valve, the tilting disc (single leaflet) valve, and the bileaflet valve are rigid structures; therefore it is not possible to collapse them to a diameter sufficient for safe implantation using small diameter catheters or delivery systems (e.g. smaller than twenty-nine french (29 f) or nine and seven-tenths millimeter (9.7 mm)).

Other prosthetic valves, such as bioprosthetic valves manufactured using pericardial tissue, are generally flexible structures, and it is possible to collapse them to a diameter sufficient for safe implantation using small diameter catheters or delivery systems. However, bioprosthetic valves cannot be readily sterilized by common heat (autoclave) or gamma radiation sterilization processes. Bioprosthetic valves typically must be sterilized with a chemical sterilant and thoroughly rinsed to remove residual chemical sterilant prior to implantation. Chemical sterilization precludes the prosthetic valve's ability to be collapsed prior to sterilization as the tightly collapsed materials would prevent the sterilant from performing its function as the sterilant would not be able to penetrate into and between the material layers. Chemical sterilization also precludes the valve from being stored in a collapsed or compressed state because significant amounts of chemical residue would remain in folds of a compressed valve even after rinsing. Thus, currently, these prosthetic valves must be stored in a non-collapsed state, necessitating compression prior to surgery.

Tissue engineered valves have also been developed but these valves tend to lack the durability required for valve replacements and also require extensive time for construction and cell seeding, all of which make them impractical for use in normal valve replacement surgery or valve function replacement procedures as patients typically present emergently.

There is a need in the art for an improved prosthetic valve having advantageous hemodynamic performance, non-thrombogenicity, and durability with the ability to be sterilized and stored in a collapsed or non-collapsed state until implantation.

### BRIEF SUMMARY

The embodiments of the invention are summarized by the claims that follow below. However, briefly, the embodiments of the invention include methods, apparatus, and systems for prosthetic valves formed with isotropic filter screen leaflets. The prosthetic valve may be applied in mammals, may use one or more flexible leaflets and may be sterilized and stored in a collapsed or non-collapsed state. An aspect of the invention is a leaflet or cusp or contiguous leaflets or cusps made from a filter screen with uniform pores having a diameter between fifteen (15) microns and sixty (60) microns that can be used to make a prosthetic valve. The filter screen material is of a biocompatible material such as polyester, polypropylene or other biocompatible material. The filter screen material may be from one-hundredth (0.01) of a millimeter to one-tenth (0.10) of a millimeter thick. The prosthetic valves may be crimped, sterilized and included in a catheter delivery system, inside a sterile barrier, ready for minimally invasive implantation without having to be rinsed prior to use.

### BRIEF DESCRIPTIONS OF THE DRAWINGS

FIG. 1 is a background FIG. illustrating a woven polyester material with non-uniform openings.

FIG. 2 is a background FIG. illustrating a woven yarn material formed with strands of yarn having non-uniform openings.

FIG. 3 is a top view of woven monofilament material with uniform openings.

FIG. 4A is a view of the woven monofilament material of FIG. 3 with a rough cut edge formed through contact method of cutting.

FIG. 4B is a view of the woven monofilament material of FIG. 3 with a smooth sealed edge formed through a non-contact method of cutting.

FIG. 5 illustrates the woven monofilament material of FIG. 3 encapsulated with smooth endothelial tissue.

FIG. 6 is a plan view of a prosthetic valve leaflet formed out of the woven monofilament material illustrated in FIG. 3.

FIG. 7 is an exploded view of assembly of a prosthetic valve with prosthetic valve leaflets of FIG. 6.

FIG. 8A is a top perspective view of a prosthetic valve with prosthetic valve leaflets of FIG. 6.

FIG. 8B is a bottom perspective view of the prosthetic valve of FIG. 8A.

FIG. 9A is a top perspective view of a prosthetic valve with three prosthetic valve leaflets in a closed condition due to back flow fluid pressure.

FIG. 9B is a top perspective view of a prosthetic valve with three prosthetic valve leaflets in an open condition due to forward flow fluid pressure.

FIG. 10A is a top perspective view of a prosthetic valve with two prosthetic valve leaflets in an open condition due to forward flow fluid pressure.

FIG. 10B is a bottom perspective view of the prosthetic valve of FIG. 10A.

FIG. 10C is a top perspective view of the prosthetic valve of FIG. 10A in a closed condition due to back flow fluid pressure.

FIG. 10D is a sectional view of the prosthetic valve of FIG. 10C in the closed condition.

FIG. 11A is a side view of a prosthetic valve with a single prosthetic valve leaflet in a closed condition due to back flow fluid pressure.

FIG. 11B is a side view of the prosthetic valve with a single prosthetic valve leaflet in an open condition due to forward flow fluid pressure

FIG. 12 illustrates an uncompressed prosthetic valve stored in a storage container.

FIG. 13A illustrates an uncompressed prosthetic valve being compressed by a radial force applied by a compression device.

FIG. 13B illustrates a compressed prosthetic valve for storage or insertion into a human being.

FIG. 13C illustrates an exploded view of a compressed prosthetic valve being stored in a storage container.

FIG. 13D illustrates a compressed prosthetic valve being stored in a storage container.

FIG. 14A illustrates a delivery system for a prosthetic valve compressed onto a balloon catheter.

FIG. 14B illustrates the balloon catheter with the compressed prosthetic valve collapsed onto a deflated balloon.

FIG. 14C illustrates the balloon catheter with the compressed prosthetic valve collapsed onto a deflated balloon being advanced inside a vessel.

FIG. 14D illustrates the balloon catheter with the balloon expanded to expand the compressed prosthetic valve into place as the uncompressed prosthetic valve.

FIG. 15A illustrates a delivery system for a compressed self-expanding prosthetic valve compressed into the distal end of catheter.

FIG. 15B illustrates the distal end of the catheter with the compressed self-expanding valve inside, being advanced along a vessel.

FIG. 15C illustrates the distal end of the catheter with the compressed self-expanding valve being ejected into the proper position inside the vessel.

FIG. 16A illustrates a compressed prosthetic valve with an anchor tab inside the distal end of a catheter.

FIG. 16B illustrates a compressed prosthetic valve with an anchor tab being pulled from the distal end of a catheter.

FIG. 17 illustrates the compressed prosthetic valve inserted into and expanded in place in a heart.

### DETAILED DESCRIPTION

In the following detailed description of the embodiments of the invention, numerous specific details are set forth in order to provide a thorough understanding of the present invention. However, it will be obvious to one skilled in the art that the embodiments of the invention may be practiced without these specific details. In other instances well known methods, procedures, and components have not been described in detail so as not to unnecessarily obscure aspects of the embodiments of the invention.

In accordance with one aspect of the present invention, a prosthetic valve is assembled from a plurality of the leaflets or cusps sewn together, or from the contiguous leaflets or cusps, described above, each having an inner face, an outer face, an in-flow edge, an out-flow edge and side edges. The leaflets are arranged so that at least a portion of their side edges form a substantially valve like structure having an in-flow end and an out-flow end. The adjacent leaflets are arranged so that their side edges are substantially aligned and the inner faces of the leaflets engage each other adjacent the side edges. The valve structure is movable between a closed position in which the out-flow edges of adjacent leaflets engage each other at a depth of between three millimeters and nine millimeters, and an open position in which the out-flow edges of adjacent leaflets are separated from each other except along the side edges so that the portions of the side edges of the leaflets bias the leaflets toward a partially closed position.

In accordance with a further aspect of the present invention, a method for making a prosthetic valve involves providing a section of substantially flat, flexible fifteen micron to sixty micron filter screen material, cutting a plurality of leaflets out of the flat material so that each of the leaflets has an inner face, an outer face, a proximal end, a distal end, side edges, and tab portions adjacent the distal end and extending from the side edges, aligning the side edges of adjacent leaflets together so that the inner faces of adjacent leaflets engage each other adjacent the side edges, and sewing aligned side edges together so as to form a substantially valve like structure having an in-flow end and an out-flow end.

Additionally, the plurality of leaflets can be formed using a non-contact cutting apparatus, such as but not limited to a laser, or a contact cutting apparatus, such as but not limited to a radio frequency (RF) or ultrasonic cutter.

In accordance with another aspect of the present invention, the leaflets of a prosthetic valve are comprised of fifteen micron to sixty micron filtration screen material made from biocompatible monofilament polypropylene, polyester, acetylpolymer (e.g. homopolymer (e.g. DELRIN®) or copolymer), or a natural material such as silk or spider web.

In accordance with another aspect of the present invention, a prosthetic valve manufactured from the leaflets comprised of a flexible fifteen micron to sixty micron screen material that can be sterilized utilizing gamma radiation, e-beam, ethylene oxide (Et0), heat (autoclave), or chemical sterilant (e.g. glutaraldehyde, hydrogen peroxide).

In accordance with another aspect of the present invention, a prosthetic valve manufactured from the leaflets comprised of a flexible fifteen micron to sixty micron pore size screen material that can be collapsed to a diameter less than or equal to twenty-nine french (29 f) or nine and seven-tenths millimeter (9.7 mm) and sterilized. This collapsed valve can be stored in a dry condition and can be implanted into a mammal without rinsing.

In accordance with another aspect of the present invention, a semilunar valve is assembled from three thin, flexible leaflets of fifteen micron to sixty micron filter pore size screen material, each having an inner face, an outer face, an in-flow edge, an out-flow edge, side edges and tab portions extending outwardly beyond the side edges and positioned adjacent the out-flow edge such that the leaflets are attached to each other along their side edges so as to form a substantially valve like structure having an in-flow end and an out-flow end. The tab portions of adjacent leaflets engage each other to form commissural attachment tabs and at least a portion of each commissural attachment tab is adjacent to the outer face of the adjacent leaflets.

Another aspect of the present invention is a method for manufacturing a prosthetic valve involving providing a first valve leaflet of fifteen micron to sixty micron filter pore size screen material and a second valve leaflet of fifteen micron to sixty micron filter pore size screen material, the leaflets being formed separately from each other, placing a portion of an inward face of the first valve leaflet against a corresponding portion of an inward face of the second valve leaflet so that they have a depth of coaptation at the outflow edge that is between three and nine millimeters, and attaching the inward face portions to each other. The inward face portions of the leaflets are attached at the side edges of the leaflets.

In accordance with another aspect of the present invention, a prosthetic valve includes a plurality of valve leaflets comprised of a flexible fifteen micron to sixty micron pore size screen material, each leaflet having an inner surface and an outer surface, each leaflet attached to another leaflet along an attachment line, a portion of an inner surface face of one leaflet being in facing relationship with a portion of an inner surface of another leaflet at the attachment line, and a commissural tab at an end of each attachment line. The tab having free ends configured for attachment to a blood vessel.

In accordance with another aspect of the present invention, a prosthetic valve includes a plurality of valve leaflets comprised of a flexible fifteen micron to sixty micron pore size screen material. The prosthetic valve may be pre-compressed for ready insertion. In one embodiment the prosthetic valve may be compressed or collapsed around a balloon catheter. In another embodiment the compressed prosthetic valve may be self expanding. The prosthetic valves may compressed, sterilized as part of a catheter delivery system or kit, inside a sterile barrier, ready for minimally invasive implantation without having to be rinsed prior to use.

### Introduction

Various authors have discussed the use of DACRON® (DuPont trademarked polyethylene terephthalate, PET, a polyester yarn (multi-filament) material similar to the material shown in background FIG. 2) whereby the DACRON® yarn is knitted or woven into a fabric and then cut to a leaflet shape. The authors discuss the desirable tissue in-growth, however the thickness of the DACRON® as well as its limited non-uniform porosity (see typical opening in DACRON® as shown in FIG. 2) prevents valves made from these types of leaflets from satisfactory performance. The thickness of this material, usually one millimeter (1.0 mm) to one and one-half millimeter (1.5 mm) or greater, does not allow for the leaflet to open and close properly during the normal cardiac cycle. As the patient's body defenses begin to encapsulate the cloth material, the leaflet thickness increases and the flexibility of the leaflet is further reduced.

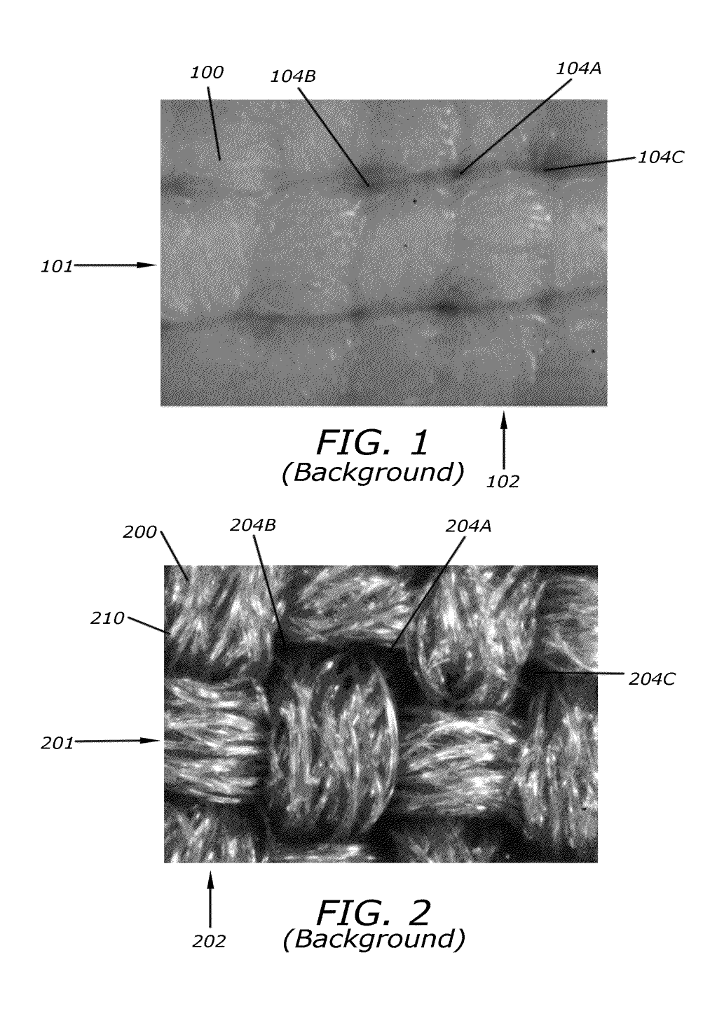

Leaflets have been formed with multilayer woven materials such as DACRON®. The DACRON® yarn is knitted or woven into a fabric and then cut to a leaflet shape. Referring now to background FIG. 1, a woven polyester material 100 is illustrated. Rows 101 and columns 102 of the polyester material are woven together to form the woven polyester material 100. At the interstices of rows 101 and column 102 are a number of small, non-uniform openings 104A-104C. The openings 104A-104C are non-uniform in size and shape. Non-uniform openings are undesirable, as tissue is unable to grow evenly through the material 100 and strengthen it across its entire surface.

A magnified view of a square of DACRON® weave 200 is shown in FIG. 2. Rows 201 and columns 202 of DACRON® yarn are woven at a substantially ninety degree angle to form a material with openings 204A, 204B, and 204C at the interstices of the rows 201 and columns 202. As more clearly shown in this magnified view, the openings 204A-C are non-uniform in size and shape. Under magnification, DACRON® is seen to be a multi-filament yarn; with each row 201 and column 202 comprising smaller fibers 210.

The thickness of woven DACRON® is usually one millimeter (1.0 mm) to one and one-half millimeter (1.5 mm) or greater. The thickness of this material does not allow for the leaflet to open and close properly during the normal cardiac cycle. As the patient's body defenses begin to encapsulate the cloth material, the leaflet thickness increases and the flexibility of the DACRON® leaflet is further reduced. The non-uniform porosity also degrades the performance of valves made from these types of materials. Non-uniform porosity promotes non-uniform encapsulation further degrading the flexibility and hemodynamic of the DACRON® leaflet over time.

While authors may have presented anisotropic, multi-layered synthetic leaflets, they have not taught that by using a single layer of isotropic material that this layer, after implantation and endothelial cell encapsulation, naturally will become one of the outer leaflet layers as the prosthetic valve integrates into the body.

### Monofilament Material for Prosthetic Valve Leaflets

Embodiments of the invention use a single layer isotropic synthetic material such as monofilament polyester, monofilament nylon, or monofilament polypropylene to form the valve leaflets.

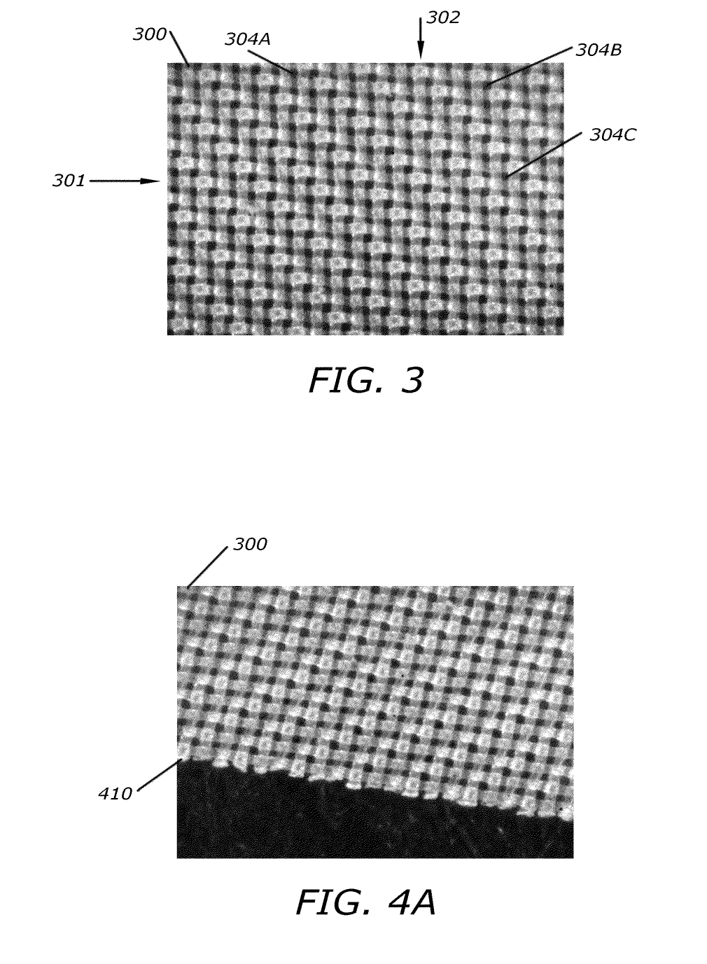

Unlike prior art material, the invention uses isotropic filtration screen as a material for a valve leaflet or leaflets. Referring to FIG. 3, one aspect of the invention uses a monofilament material 300 composed of monofilament strands 301,302 woven or knitted into a single layer of isotropic material. A cross section shape of the monofilament strands 301,302 may be round, oval, square, or rectangular, for example. The openings or pores 304A-C at the interstices of the monofilament strands 301,302 have a pore size that allows fifteen (15) micron to sixty (60) micron particles to pass through them. Accordingly, a pore size between monofilaments for the material 300 may be in the range between fifteen (15) microns to sixty (60) microns, inclusively. For example, the pore size of the openings or pores 304A-304C in accordance with one embodiment of the invention are approximately twenty-seven (27) microns. Usually red blood cells are about six (6) microns to eight (8) microns in diameter and approximately two microns thick, thus they are capable of passing through pores 304A-C with a pore size of twenty-seven (27) microns relatively intact.

Unlike DACRON®, the openings 304A-304C of monofilament material 300 are generally of uniform size. With uniform openings, tissue in-growth or endothelialization occurs uniformly throughout and provide a strengthening layer over the entire surface. Additionally, the smaller thickness or cross-section distance of the monofilament fiber 301, 302 which forms the uniform openings 304A-304C allow the material 300 to flex readily between a concave surface and a convex surface with little to no damage to the material 300.

The thinner single layer of monofilament material 300 also promotes faster endothelialization. At about one-tenth the thickness of DACRON®′ the monofilament material 300 is less likely to induce calcification as the amount of material will be substantially encapsulated by native tissue. Endothelization may also promote better hemodynamics and reduce thrombogenicity and rejection of the prosthetic valve.

Monofilament material 300 is also more resilient than some of the prior art materials. For example, after opening and closing approximately forty million times, solid sheet materials such as MYLAR® may develop a crease from plastic deformation of the material, eventually leading to valve failure. The smaller monofilament strands making up monofilament material 300 have a smaller critical radius thus they are more resistant to the effects of plastic deformation. The small cross section distance of the monofilament strands 301, 302 which forms the uniform openings 304A-304C allow the monofilament material 300 to flex readily between a concave surface and a convex surface with little to no damage to the monofilament material 300.

Unlike natural or cross-linked (e.g. glutaraldehyde fixed) tissue, the monofilament material 300 may be readily sterilized by heat and gamma radiation. Bioprosthetic tissue is generally limited to chemical sterilization such as by two to four percent glutaraldehyde. Thus, the monofilament material 300 is the preferred choice of material for a prosthetic valve leaflet.

The monofilament strands 301,302 are made from biocompatible polypropylene, polyester, silk, spider web, or other biocompatible material. The thickness of the woven or knit material is between one hundredth of a millimeter (0.01 mm) and one-tenth of a millimeter (0.10 mm). According, the cross section distance of a monofilament strand may be in the range of one hundredth of a millimeter (0.01 mm) and one-tenth of a millimeter (0.10 mm), inclusively. The screen material fabricated from the monofilament strands 301,302 may be cut into leaflets.

A leaflet may be cut from the filter screen material using a non-contact method such as a laser to ensure that the cut edges of the leaflet do not substantially have any extending fibers. The leaflet could also be cut from the material using a heat contact method, such as with a radio frequency (RF) or ultrasonic cutter, as long as the edges of the finished leaflet do not substantially have any extending fibers. Using the non-contact or heat contact methods described ensures that the edges of the leaflet are fused and prevents the leaflet from coming apart or the monofilaments from unraveling. The leaflet will be less thrombogenic as there will be no fibers extending out from the edge of the leaflet that would prompt a thrombogenic response from the body.

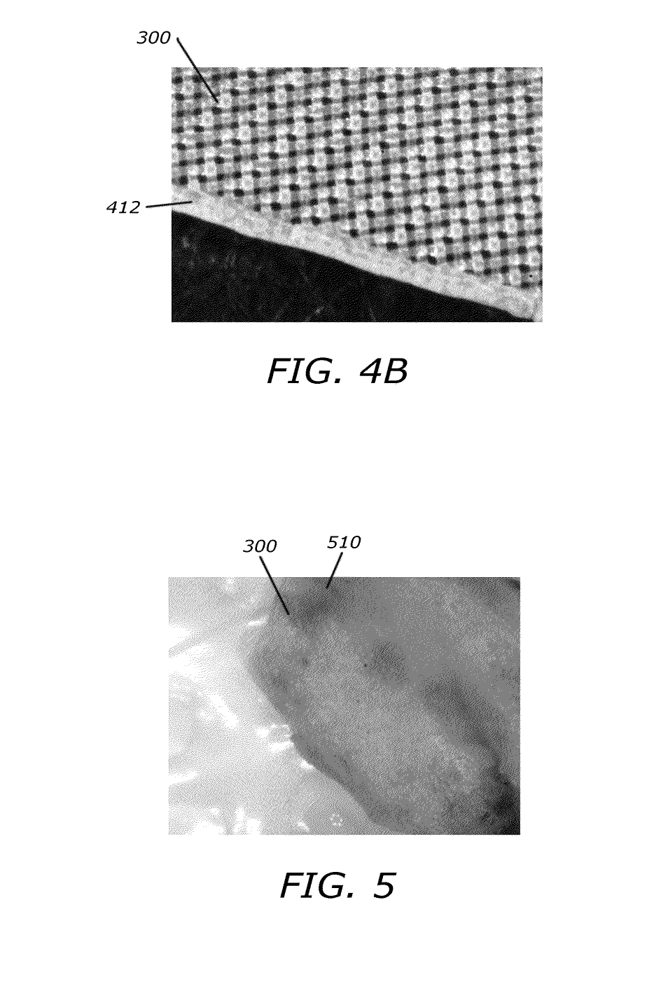

Referring now to FIG. 4A, an edge 410 is illustrated in the monofilament material 300 formed by a shearing method of cutting. As a shearing method of cutting was used, the edge in the monofilament material 300 is a rough-cut edge 410. A rough-cut edge 410 edge may be satisfactory when the monofilament material 300 is coupled to another surface. A suture, for example, may deter the woven monofilament material from unraveling in the leaflet at the rough cut edge 410. The rough cut edge 410 may also be heated until the rows and columns at the edge melts. Melting the edge may fuse or seal the rows and columns of monofilament strands together to keep them from unraveling. Typically, at least one edge of the leaflet is an open edge (e.g. outflow edge) that is preferably sealed to avoid unraveling.

Referring now to FIG. 4B, a noncontact cutting or cutting method may be used to cut an edge in the monofilament material 300. A laser is an example of a noncontact method of cutting the material, while an ultrasonic, radio frequency (RF) cutter or heated blade or die may be an example of a contact cutting method. Both cutting methods leave a smooth sealed or fused edge 412 in the monofilament material 300. The prosthetic valve leaflet may have one or more of its edges cut in this manner so that one or more respective edges are a smooth sealed edge 412.

The monofilament material, after being cut into a valve leaflet, may be coated with a collagen material that surrounds and penetrates into the monofilament screen to form a mechanical lock. The monofilament material acts as a scaffold for cell growth in-vivo allowing mammalian autologous cells to adhere to, and to grow on, the scaffold. This growth occurs naturally after implantation in the mammal thereby creating a smooth, highly biocompatible, non-thrombogenic surface.

Referring now to FIG. 5, a top view of tissue 510 grown into the monofilament material 300 is illustrated. To seal the openings 304A-304C within the monofilament material 300, tissue 510 is allowed to grow into and around the columns and rows of the monofilaments. In FIG. 5, the smooth endothelial tissue 510 substantially encapsulates the monofilament material 300. After twelve to forty-eight hours of exposure to in-vivo blood flow, the smooth endothelial tissue 510 can substantially encapsulate the monofilament material 300. In one embodiment, for example, a twenty-seven micron monofilament material was substantially encapsulated by smooth endothelial tissue 510 after less than twelve hours.

### Prosthetic Valve Leaflets

A leaflet made from the material 300 described above presents to the blood flow a surface that is initially porous and will allow blood components such as red blood cells to pass through it. While a red blood cell will pass through the screen material, the screen still presents a restriction to flow much like a window screen partially blocks air flow through a window. When these leaflets are used to form a prosthetic valve, this type of screen material in the blood flow will initially prevent lysis, or destruction, of the red blood cells when the leaflet sections close, but still provide enough resistance to the blood flow to act as a one-way valve.

As the body reacts to the material 300, endothelial cells will form on the surface of the screen and gradually grow on and anchor themselves through the screen material. This endothelialization will gradually reduce the screen porosity with the patient's own native cells and the porosity of the screen will naturally decrease and the valve will become more competent and less regurgitant. Over time, it is expected that the encapsulated leaflet will begin to take the shape of a natural leaflet and the layer of screen material will take the role of an outer, less elastic layer of the leaflet while the patient's own tissue will develop into the other layers such as the inner more elastic layer.

Endothelization may also improve the hemodynamics and reduce the thrombogenicity of the prosthetic valve. The smooth endothelial tissue reduces fluid resistances through the valve. Also, because the endothelial layer is formed of the patient's own native cells, there is less likelihood of implant rejection.

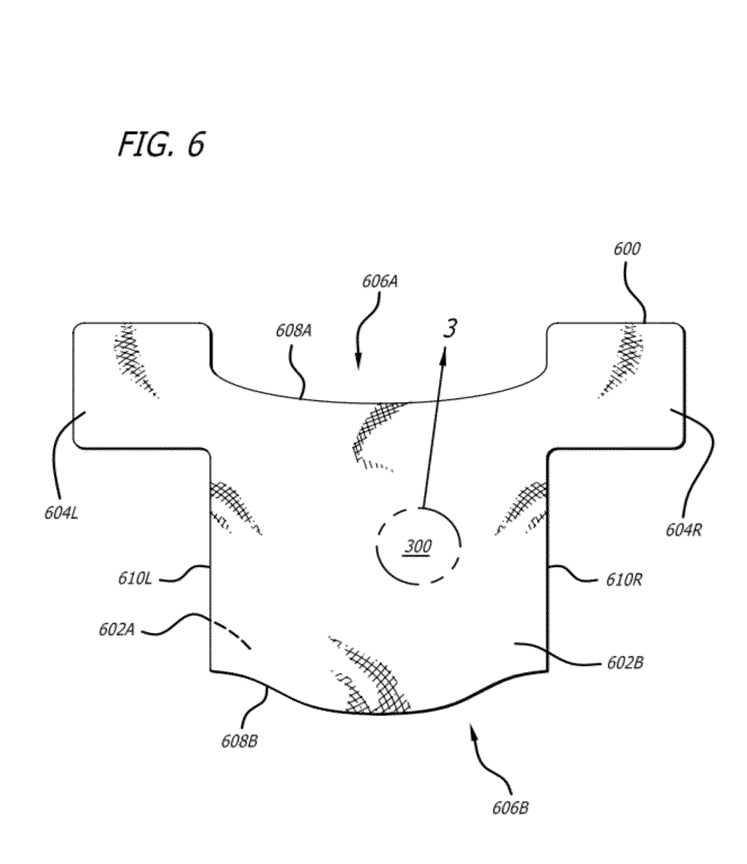

Referring now to FIG. 6, an example of a prosthetic valve leaflet 600 formed out of the woven monofilament material 300 is illustrated. The monofilament material 300 is cut into the shape of the prosthetic valve leaflet 600. One or more of the edges of the valve leaflet are cut using the noncontact cutting or heat cutting method to provide a smooth, sealed edge. The leaflet can also be referred to herein as a cusp.

The exemplary prosthetic valve leaflet 600 has an inner surface 602A and an outer surface 602B. The leaflet 600 further has an inflow end 606B and an outflow end 606A. Near the inflow end 606B, an inflow edge 608B is cut into the material 300. Near the outflow end 606A, an outflow edge 608A is cut into the material 300. A left-side edge 610L and a right-side edge 610R, forming a pair of side edges 610, are cut into the material 300 to further form the valve leaflet 600. A left-tab portion 604L and a right-tab portion 604R are also cut into the material 300 to form a pair of tab portions 604 in the leaflet 600.

One or more of the prosthetic valve leaflets 600 may be used to form a prosthetic valve to be inserted within a mammalian body. The prosthetic valve functions as a passive one-way check valve. In the case of three leaflet valves, an adjacent left tab and right tab of adjacent leaflets are coupled together to form a joint tab. Fluid flow in the opposite direction of the prosthetic valve may cause the outflow edges 608A of prosthetic valve leaflets 600 to push together and engage, thereby blocking the regurgitant flow. Similarly, two valve leaflets may have their tabs coupled together to form a check valve where regurgitant flow may also cause the outflow edges 608A to push together and engage to close the valve. In another embodiment with a single valve leaflet 600, the tab 604A or 604R and inflow edge 608B may be sewn to a vessel sidewall while the outflow edge 608A flaps open and closed against an opposite vessel sidewall.

While FIG. 6 illustrates one shape of a prosthetic valve leaflet 600 that may be cut from the isotropic screen material, other shapes for a valve leaflet may cut as well and used in a prosthetic valve. For example, other shapes of valve leaflets are disclosed in U.S. Pat. No. 2,822,819 issued to Geeraert; U.S. Pat. No. 3,197,788 issued to Segger; U.S. Pat. No. 4,297,749 issued to Davis; U.S. Pat. No. 4,388,735 issued to Ionescu; U.S. Pat. No. 4,470,157 issued to Love; U.S. Pat. No. 4,501,030 issued to Lane; U.S. Pat. No. 6,338,740 issued to Carpentier; U.S. Pat. No. 6,454,799 issued to Schreck; U.S. Pat. No. 6,893,460 issued to Spenser; and U.S. Pat. No. 7,044,966 issued to Svandize et al; as well as US Pat. App. Pub. Nos. 2004/0225356 by Frater, 2006/0235511 by Osborne, 2007/0270944 by Bergheim et al, and 2010/0174361 by Bailey et al.; all of which are incorporated herein by reference.

### Prosthetic Valves for Mammals

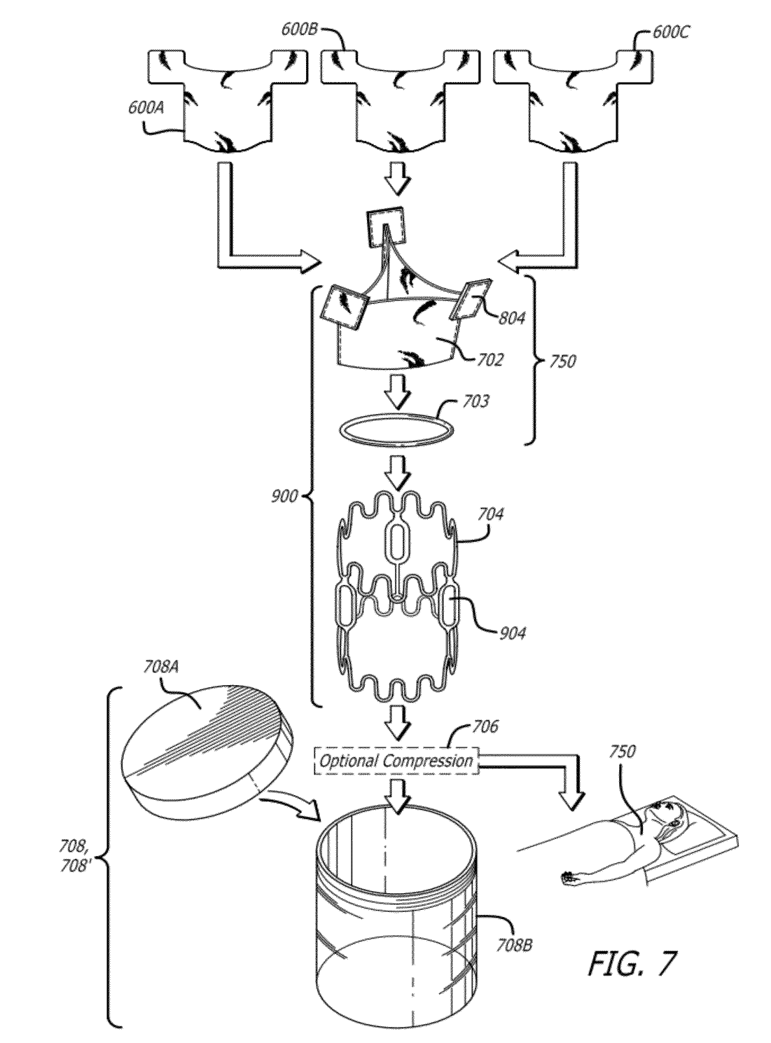

Referring now to FIG. 7, an exploded view of the assembly of a prosthetic valve utilizing three leaflets 600 is illustrated. The three leaflets 600A-600C are coupled together to form a tri-leaflet structure 702. The adjacent tabs of each leaflet 600A-600C are coupled together to form joint tab 804. A left edge 610L is coupled to a right edge 610R of an adjacent leaflet (refer back to FIG. 6). A sewing ring 703 formed of the monofilament material 300 may be formed and coupled to the tri-leaflet 702 along each outflow edge 608B. Although depicted as a separate piece, sewing ring 703 may be unitarily formed from the inflow edges of the tri-leaflet structure 702, e.g. by rolling the edges. Sewing ring 703 may be used to anchor the inflow edge of the prosthetic valve, for example by coupling the sewing ring 703 to the inner wall of an outer frame or stent or directly to an artery.

With the tri-leaflet structure 702 and the sewing ring 703 coupled together, a first embodiment of a prosthetic valve 750 is formed. The prosthetic valve 750 may be sewn directly to an artery or other tissue or may be further coupled to a frame 704, for example. The valve 750 may have joint tabs 804 inserted into tab openings 904 within the frame 704. The valve 750 is inserted into the inner hollow area of the frame 704, to form another embodiment of a prosthetic valve 900.

Frame 704 may be constructed of a variety of metals and polymers so long as the material is flexible, supportive, capable of being collapsed and subsequently expanded (if applicable), and biocompatible. Stainless steel, gold, titanium, cobalt-chromium alloy, tantalum alloy, Nitinol (Nickel Titanium Naval Ordinance Laboratory) are examples of metals used in stent frames. Nitinol is an ideal metal because it is highly biocompatible, corrosion resistant, very flexible and has excellent shape memory when heated to a certain temperature. Shape memory allows a Nitinol stent to be cooled, compressed, and retained in a compressed state, and surgically inserted. As the Nitinol warms, it will return to its uncompressed state without deformation.

Certain polymers such as acetylpolymer, polypropylene, silicone, polyethylene and polyurethane have also found use as stent materials. The number and type of polymers developed for use in medical devices is expanding as different polymer types, chemistries and manufacturing processes are used to produce devices or device coatings with a wide variety of functional characteristics. Shape-memory polymers can also be used to produce a device that will transition from a temporary state to a different (permanent) state through the inducement of a stimulus of heat or cold.

The valve 750, 900 is compressible by an optional compression step 706. The valves 750, 900 can be stored in a container for later insertion into a body. The valves 750, 900 after assembly can be compressed by the optional compression 706 and inserted into a body 750. Alternatively, the valves 750, 900 may be stored in containers 708, 708′. The container includes a base 708B with one closed end and one open end, and a cap 708A to close over the open end. If the valves 750, 900 are compressed by the optional compression step, the storage container 708′ may be used as it is smaller and more compact than the storage container 708. The storage container 708 is sized to store the non-compressed valve.

In one embodiment of the invention, the prosthetic valve 900 is compressed and included in a delivery system or kit, such as a catheter delivery system. Alternatively, the prosthetic valve 900 is compressed and coupled to the catheter and included as part of the delivery system. In either case, the collapsed valve is sterilized and stored in the collapsed state.

Pre-compression and delivery in a sterilized state 708,708′ may be advantageous as a timesaving measure and safety measure. A valve delivered in a sterile compressed state is ready for insertion without the surgeon needing to manually compress, and possibly damage or contaminate the valve. Prosthetic valve 750,900 may be compressed and subsequently sterilized during the manufacturing process using gamma radiation, e-beam, ethylene oxide (Et0), heat, or chemical sterilization. Bioprosthetic tissue valves cannot be readily sterilized with heat or gamma radiation without damaging the cross-linked tissue of the valves. Bioprosthetic valves may be delivered in a chemical sterilant, however bioprosthetic valves cannot be safely sterilized and stored pre-compressed. The chemical sterilant cannot readily penetrate into and between the layers of the compressed bioprosthetic valve and the chemical sterilant must be rinsed off before valve insertion, and a bioprosthetic valve in a compressed state has recesses or folds in the leaflets that would prevent adequate rinsing. Therefore, an advantage of using material 300 to form the leaflets is delivery of a pre-compressed sterile valve ready for surgical insertion.

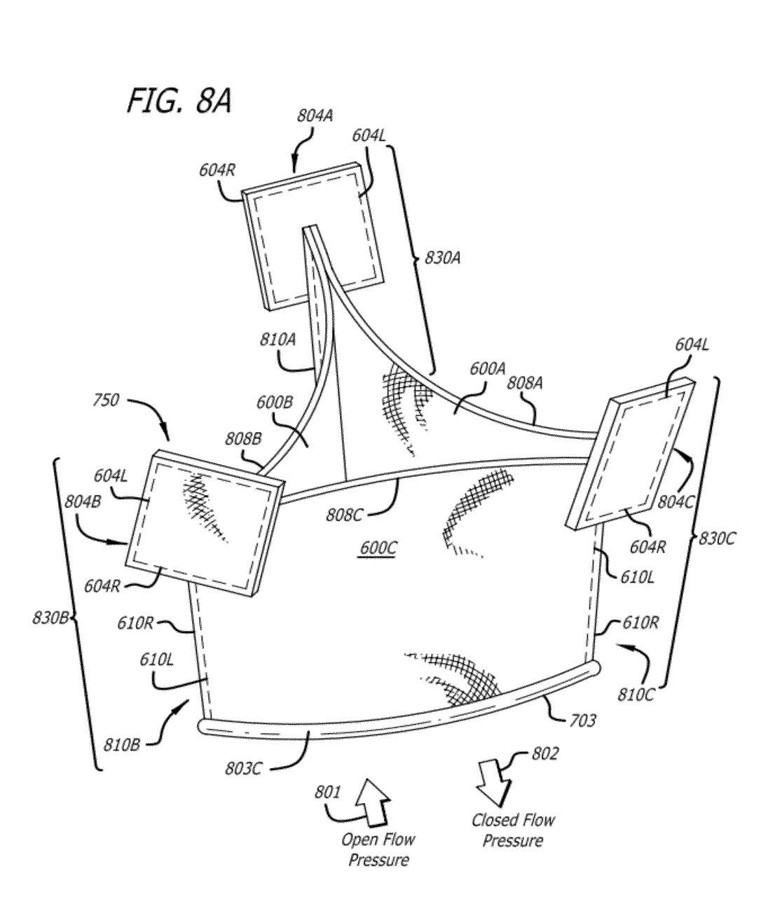

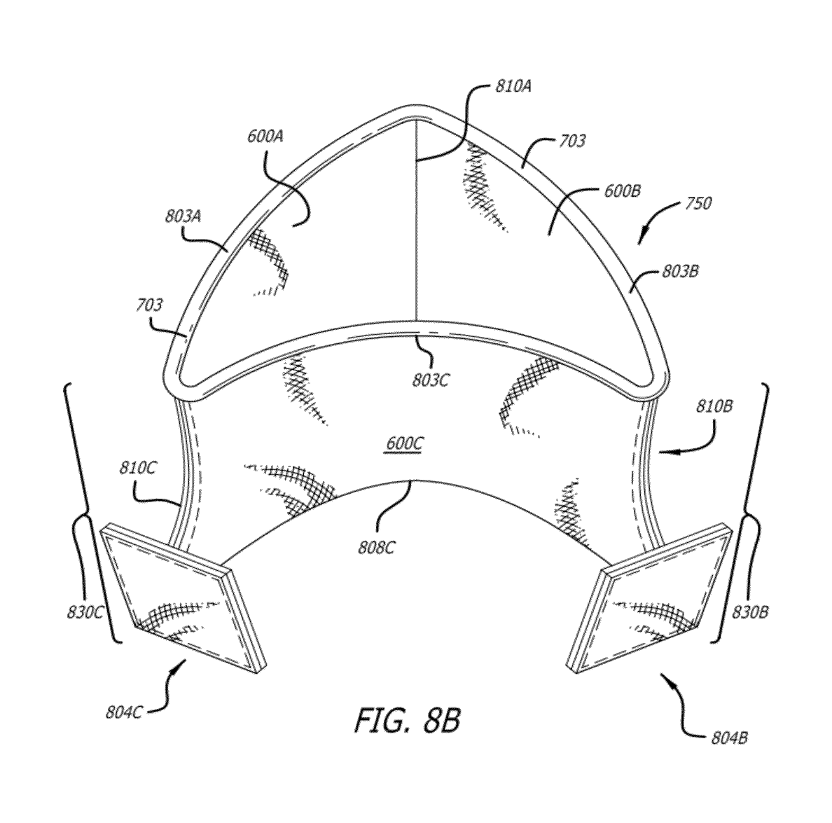

Referring now to FIGS. 8A-8B, further details of the tri-leaflet prosthetic valve 750 are now described. FIG. 8A illustrates a top view of the tri-leaflet prosthetic valve 750, while FIG. 8B illustrates a bottom view of the tri-leaflet prosthetic valve 750. The prosthetic valve operates like a check valve. The tri-leaflet prosthetic valve 750 is opened when open flow pressure 801 is experienced in one direction. The tri-leaflet prosthetic valve 750 is closed when a closed flow pressure 802 is experienced in the opposite direction. During an open flow pressure 801, the outflow edges 808A-808C of each of the respective leaflets 600A-600C are pushed away from each other so that the valve remains open. In the case of closed flow pressure, the edges 808A-808C and upper part of the face of each leaflet 600A-600C respectively, close against each other engaging to seal during a backflow of fluid, generating the closed flow pressure 802.

Left-tab portion 604L of one leaflet and a right-tab portion 604R of an adjacent leaflet are coupled together to form a joint tab 804A-804C, collectively referred to as joint tabs 804. Similarly, left side edge 610L of one leaflet is coupled to the right side edge 610R of an adjacent leaflet to form side edges 810A-810C of the valve 750. Together, the joint tabs 804 and the side edges 810 form commissure portion 830A-830C as illustrated in FIG. 8A. Collectively, the three commissure portions 830A-830C may be referred to herein as commissure portions 830. Although this embodiment is depicted comprising joint tabs 804, it should be known that joint tabs are optional to the functionality of prosthetic valves. Thus, the commissure portions 830 may comprise only side edges 810 without deviating from the scope of the invention. The inflow edge 608A of each leaflet 600A-600C is coupled to the sewing ring 703 such as shown by the edge 803C in FIG. 8A. FIG. 8B better illustrates the inflow edge of each leaflet 600A-600C coupled over the sewing ring 703.

As mentioned previously, the valve 750 may be directly sewn into an artery or other flow vessel within a mammalian body. However, to provide further support structure for the valve 750, a frame or stent may be provided as previously discussed.

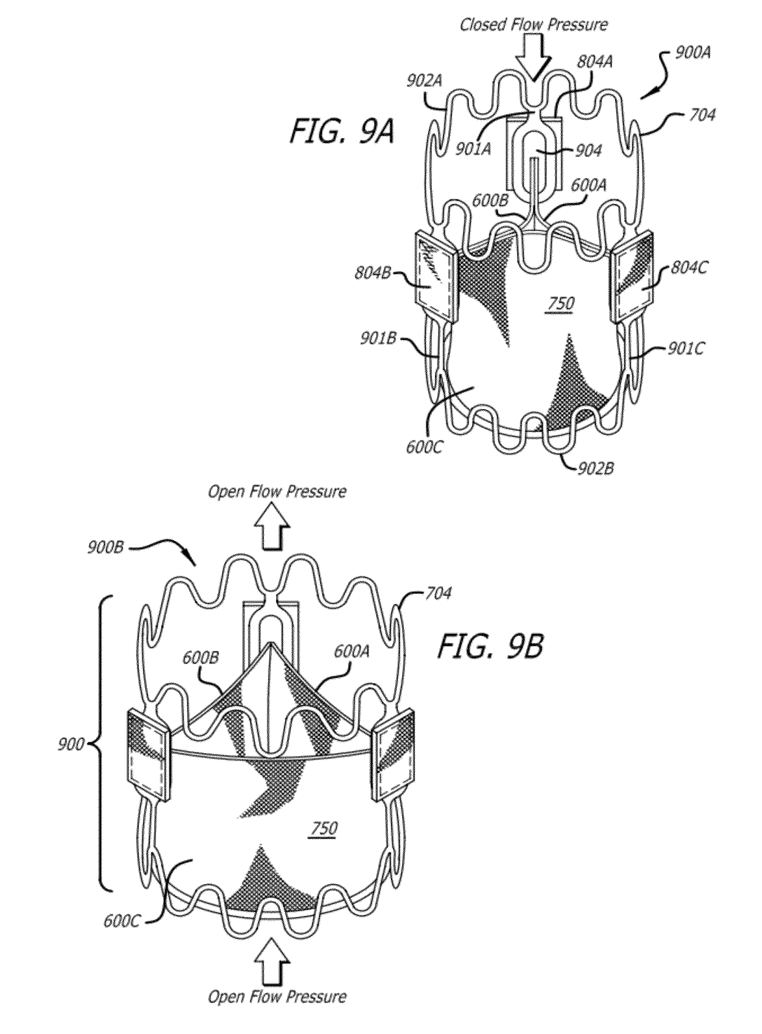

Referring now to FIGS. 9A-9B, the valve 750 is illustrated coupled to the stent/frame 704 to form the valve 900. The valve 750 is inserted inside the hollow structure of frame 704. Joint tabs 804A-804C of the valve 750 may be inserted into tab openings 904A of the frame 704. The valve 900 is in a generally closed position 900A as shown in FIG. 9A. The valve 900 is in a generally open condition 900B as shown in FIG. 9B.

The frame/stent 704 includes compressible S-shaped rings 902A-902B coupled together by two or more struts 901A-901C. Each strut 901A-901C includes a tab opening 904 for attachment of the joint tab 804A-804C of the tri-leaflet valve 750. The compressible S-shaped rings 902A-902B can be compressed so that the circumference and diameter of the valve 900 may be reduced. Once compressed the valve can be stored in a more compact state and ready for insertion into a body. If the frame/stent is constructed of a shape memory metal or polymer, the compressed state can be maintained by cooling the valve 900 after compression or by using some form of mechanical restraint.

FIG. 9A illustrates valve 900 experiencing a closed flow pressure such that each of the valve leaflets 600A-600C have been pushed together to engage at the outflow edge. The resulting coaptation at the outflow edge, seals the valve so that minimal fluid can leak through the closed valve 900A.

FIG. 9B illustrates an open flow pressure pushing through the inflow end of the valve 900. The inflow pressure passing though the internal portion of the valve flexes each leaflet 600A-600C so that the outflow edge of each is pushed open. Fluid is allowed to pass freely through the opened valve 900B.

As mentioned herein, one or more valve leaflets 600 with the monofilament material 300 may be used to form a prosthetic valve. FIGS. 7, and 8A-8B, and 9A-9B, illustrate three valve leaflets 600A-600C being used to form a prosthetic valve 750,900 with the monofilament material 300. However, two valve leaflets 600 may also be used to form a prosthetic valve with the monofilament material 300.

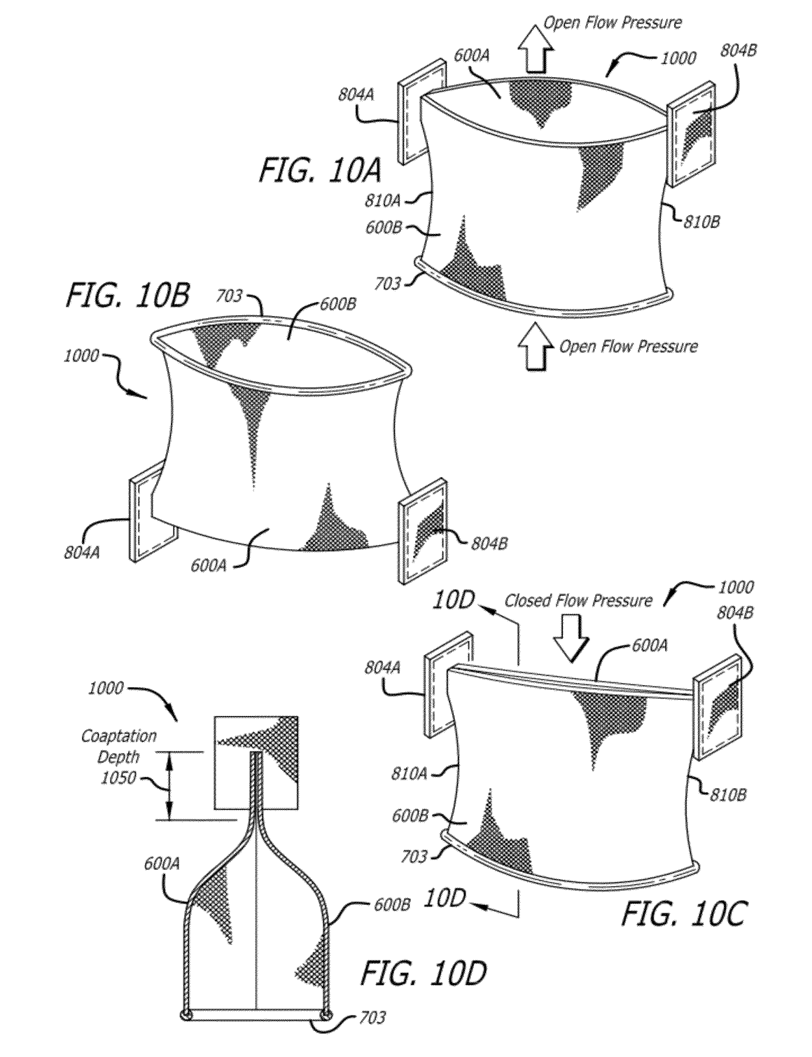

A two leaflet valve may be manufactured by forming a first and second valve leaflet of 15 to 60 micron filter screen material. The two leaflets are placed together with a portion of an inward face of the first valve leaflet against a corresponding portion of an inward face of the second valve leaflet so that they have a depth of coaptation that is between three and nine millimeters. The inward face portions are then attached to each other side edges of the leaflets by sewing or other form of adhesion. Exemplary embodiments of two leaflet valves are illustrated in FIGS. 10A-10D.

Referring now to FIGS. 10A-10B, a pair of valve leaflets 600A-600B formed of monofilament material 300 are illustrated coupled together to form a prosthetic valve 1000. The prosthetic valve 1000 includes the valve leaflets 600A-600B coupled together to form joint tabs 804A-804B, a left-side edge 810A, and a right-side edge 810B. The prosthetic valve 1000 further includes a sewing ring 703 coupled to each of the valve leaflets 600A-600B.

A top view and a bottom view of the prosthetic valve 1000 are respectively shown. FIGS. 10A-10B illustrate the prosthetic valve 1000 in an open condition. FIGS. 10C-10D illustrate the prosthetic valve 1000 in a closed condition.

In FIG.10A the sewing ring and inflow edges are depicted at the bottom of the illustration. The open flow pressure flows from the bottom into the sewing ring 703, through valve body, and out the top of the valve at the outflow end. The open flow pressure pushes the outflow edges apart allowing fluid to freely pass through the valve 1000.

In FIG. 10B the sewing ring and inflow edges are depicted at the top of the illustration. The open flow pressure flows through the sewing ring, valve body, and outflow end in the same manner as FIG. 10A although from an opposite perspective.

In FIG. 10C, the closed flow pressure at the top of the illustration pushes prosthetic valve 1000 into a closed valve condition. The closed flow pressure pushes back against the outflow edges of each valve leaflet 600A-600B. The closed flow pressure against the outflow edges forces the valve leaflets 600A-600B to flex towards each other and form a concave surface such that the surface of each further couple together to close the valve and shut off fluid flow. A clearer depiction of the closed state of an exemplary valve 1000 is illustrated in the side view of FIG. 10D.

Referring now to FIG. 10D, the valve leaflets 600A and 600B are experiencing a closed flow pressure which has pressed them together such that their outflow edges have engaged. The closed flow pressure pushes on the concave portion of the valve leaflets 600A-600B, causing the outflow edges to first touch and then coapt. The monofilament material 300 is flexible so that not only the edges merge together but further portions of the valve leaflet merge together, such that a coaptation depth 1050 is formed. Not only is the material 300 flexible, it is also strong and can withstand cycles of flexing between a concave and a convex surface without breaking.

The contact between edges where the leaflets are in opposition to each other is usually referred to as the coaptive edge, coaptive depth, edge of coaptation, depth of coaptation or simply coaptation. The valve comprised of the filter screen leaflets will be constructed in such a manner so that the coaptive depth is between three millimeters to nine millimeters but ideally about six millimeters to eight millimeters. A coaptive depth allows the edges of each leaflet to properly support the other leaflet. A larger coaptive depth distributes the closing force over a greater contact surface and reduces damage to the outflow edges of the leaflets. After the endothelization of the leaflets, this depth of coaptation prevents the delicate layers of cells from being damaged as the edges of the leaflets essentially experience lower tensile forces and the closing forces are carried over a larger surface area. The bulk of the force is handled by the leaflet surface structure at the point of coaptation.

Referring now to FIG. 11A and 11B, a valve 1100 is depicted within a vessel such as a vein 1102 or other type of cylindrical-like (cylindrical or semi-cylindrical) shaped vessel. The valve 1100 comprises a single valve leaflet 600. The leaflet 600 has an edge coupled to the inner wall 1104 of the vessel 1102 by a suture 1101, for example.

FIG. 11A illustrates the valve 1100 in a closed position due to closed flow pressure. The valve leaflet 600 extends out and presses up against the wall 1104 of the vessel 1102. The valve leaflet is longer than the diameter of the vessel 1102 and forms a coaptive depth 1150 with the vessel wall 1104. As with the coaptive depth along the edges of opposing leaflets, the coaptive depth 1150 distributes the closing force over a greater contact surface and reduces damage to the outflow edge of the leaflet 600.

FIG. 11B illustrates the valve 1100 in an open position due to open flow pressure. The leaflet 600 has flexed into a position 600′, as illustrated in FIG. 11B, to allow fluid flow past the valve 1100.

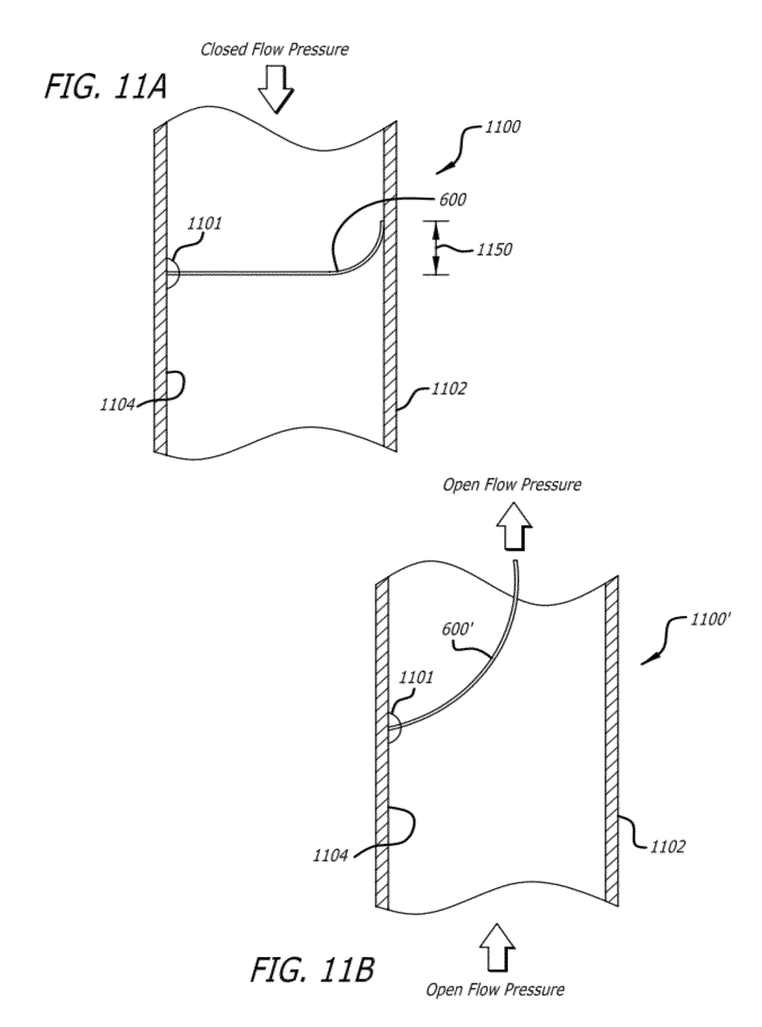

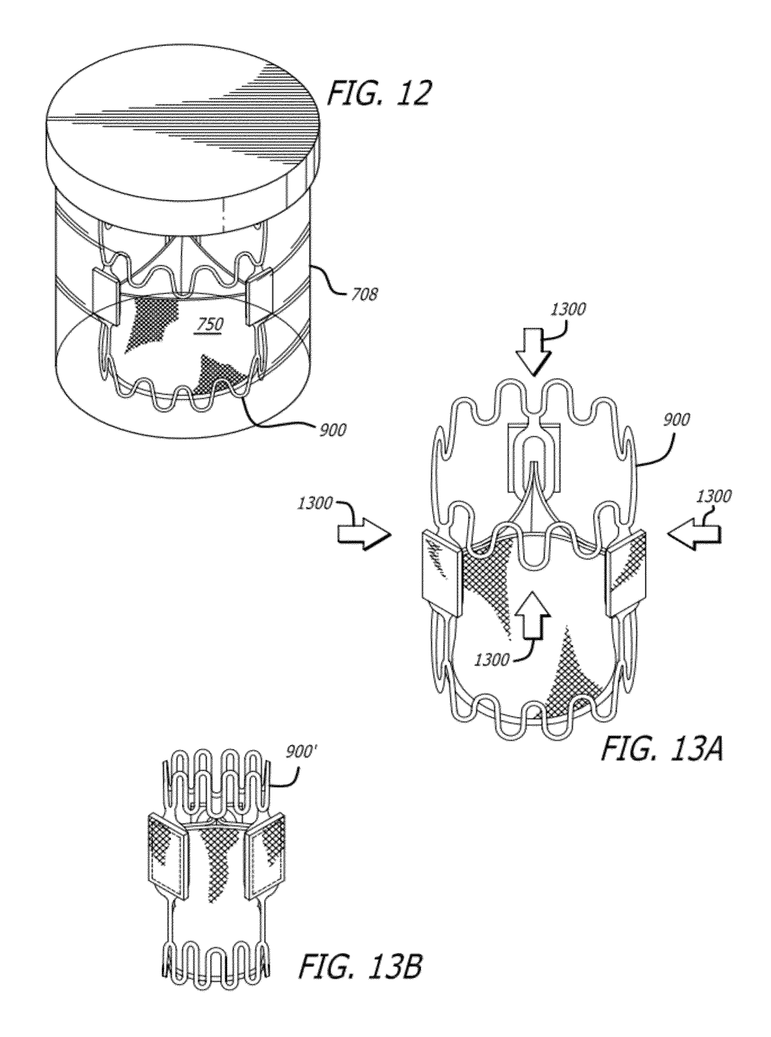

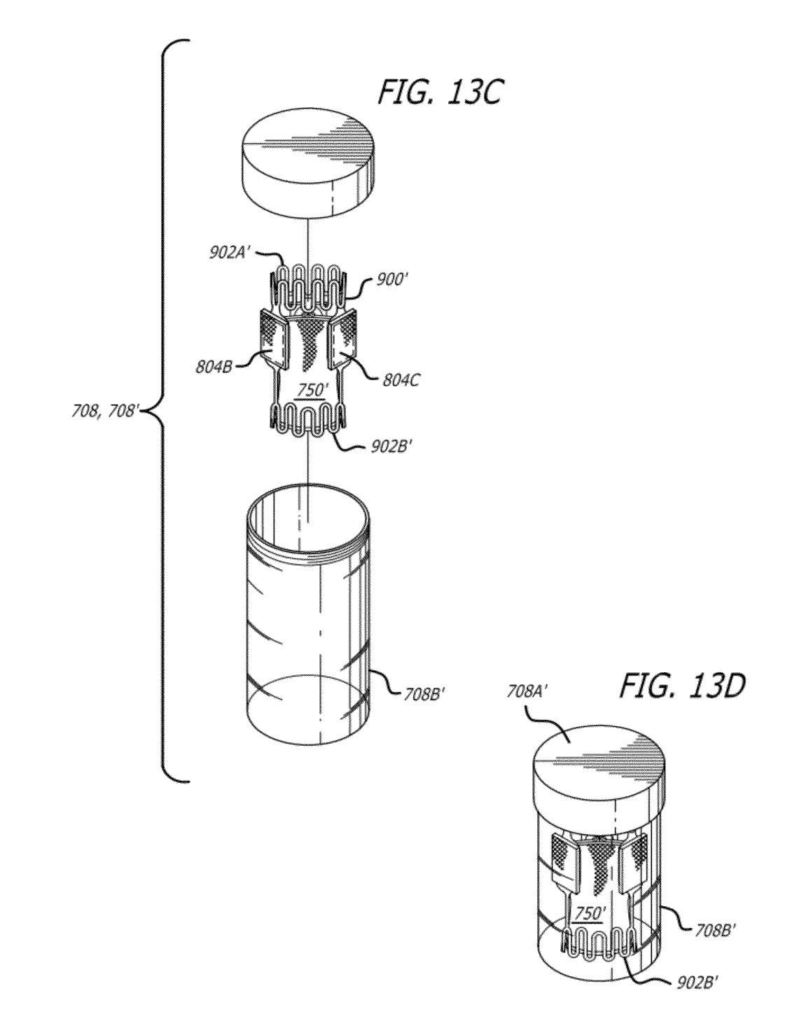

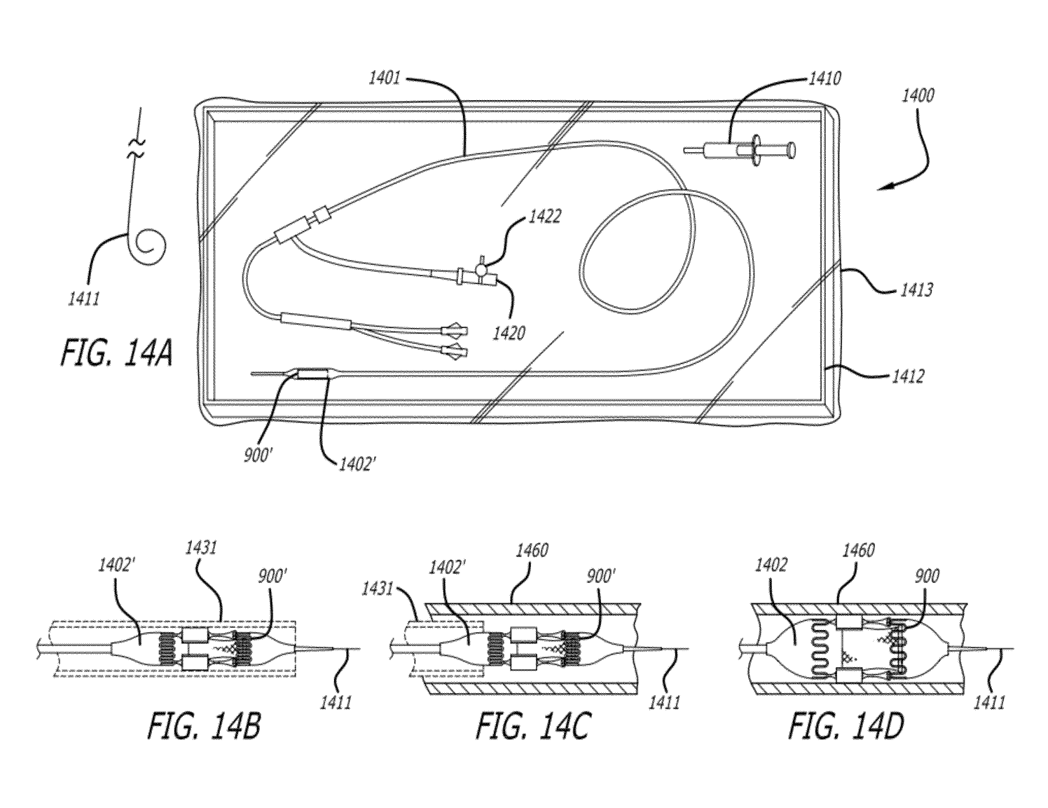

Referring now to FIG. 12, the valve 900 may be stored in a storage container 708 in an uncompressed condition ready for surgical insertion. Alternatively, the valve 900 can be compressed into a compressed valve 900′ and stored in a compression storage container 708′, such as illustrated in FIG. 13C. The valves 900,900′ in the storage containers 708,708′ may be included as part of a delivery system kit. Alternatively, the valve 900 can be compressed and removably coupled to a catheter as part of a delivery system kit (e.g., see FIGS. 14A-14D,15A-15D, and 16A-16B and the description thereof).. An uncompressed valve 900 may be compressed to a compressed valve 900′ prior to surgery. To compress the valve 900, a force 1300 is typically radially applied to the valve 900 to compress it to the compressed valve state 900′, such as shown in FIGS. 13A-13B.

The diameter of the storage container 708′ is significantly smaller than the diameter of the storage container 708. The compressed valve 900′ has its compressible S-shaped rings 902A-902B compressed into a compressed state as illustrated by the compressed rings 902A′-902B′ in FIG. 13C. The valve 750 is also compressed along with the stent/frame to a compressed state 750′.

Compression storage container 708′ may have a diameter sufficiently narrow to physically keep the compressed valve 900′ in a compressed state during ambient temperature shipment and storage. Prior to surgical insertion, if the stent is constructed of a shape memory material, the compressed valve 900′ may be subjected to cool temperatures to retain the compressed state once removed from the compression storage container 708′ prior to loading into a delivery system. It may be advantageous to keep the valve 900 in a compressed state so that the surgeon does not have to recompress the valve 900 prior to surgical insertion.

### Catheter Delivery Systems and Kits

Minimally invasive medical procedures are aimed at reducing the amount of extraneous tissue damage during diagnostic or surgical procedures, thereby reducing patient recovery time, discomfort, and deleterious side effects. The average length of a hospital stay for a standard surgery may also be shortened significantly using minimally invasive surgical techniques. Patient recovery times, patient discomfort, surgical side effects, and time away from work may be reduced with minimally invasive surgery.

One type of minimally invasive medical procedure uses a catheter to deliver a prosthetic valve to a site within a body channel. For example, in percutaneous aortic valve replacement or Transcatheter Aortic Valve Implantation (TAVI), a replacement valve compressed on a balloon catheter is passed through a hole in the groin via a puncture of the femoral artery and advanced up to the ascending aorta of the patient. The replacement valve is positioned directly inside the diseased aortic valve and the balloon is inflated to secure the valve in place. The transfemoral approach may be performed with general anesthesia and may be preferable for patients who are not candidates for open chest surgery due to age or infirmity.

A balloon catheter may be used with a compressed valve mounted on the balloon. A tubular catheter may be used that constrains a compressed self expanding valve to deliver the valve to a desired location. Example delivery systems include US Pat. Nos. 5,840,081 issued to Andersen et al.; U.S. Pat. No. 6,682,558 issued to Tu et al.; U.S. Pat. No. 6,893,460 issued to Spenser et al.; and U.S. Pat. No. 8,016,877 issued to Seguin et al. as well as US Pat. App. Pub. Nos. 2004/0225354 by Allen et al.; 2007/0239269 by Dolan et al.; 2007/0027534 filed by Bergheim et al.; 2009/0192585 by Bloom et al., 2009/0192586 by Tabor et al.; 2010/0217371 by Noone et al.; 2010/0217385 by Thompson et al.; 2010/0234940 by Dolan; 2011/0202128 by Duffy; 2011/0251679 by Wiemeyer et al.; 2011/0257733 by Dwork; 2011/0264198 by Murray III et al.; and 2011/0264203 by Dwork et al.; all of which are incorporated herein by reference.

Referring now to FIG. 14A-14D, a compressed prosthetic valve 900′ may be included as part of a delivery system or kit 1400. In one embodiment, the kit 1400 may include a catheter 1401 with a valve 900′, an inflation syringe 1410, a tray 1412, a guide wire 1411, and a container 1413. A collapsed or compressed valve 900′ is located at the distal end of the catheter 1401, ready for insertion and deployment. Unlike prior art systems, the compressed prosthetic valve 900′ is ready to be implanted without having to be rinsed, compressed, sterilized, or placed on a catheter.

The container 1413, may be a bag, box, case, tray with a lid or other sealed package to contain the catheter 1401 and valve 900′, the inflation syringe 1410, and optionally the guide wire 1411 and the tray 1412. The sealed container 1413 provides a sterile barrier to maintain the contents in a sterile state until they are to be used in surgery. The sterile barrier isolates the sterile devices inside from the non-sterile world outside. The container 1413 may be a solid plastic bag, Tyvek or paper bag or combination of solid plastic and Tyvek or paper bag. It may also be a tray with a Tyvek or paper “lid” sealed to it or a tray with a lid taped to or otherwise adhered to the tray. The tray may be wrapped with a paper wrap (e.g. sash wrap) that is placed around the tray. Sash wrap is commonly used to wrap a stainless steel tray with a lid before autoclaving and prevents the tray and lid from opening during sterilization and transport. The outer wrap creates a “torturous path” that prevents organisms from penetrating into the sterile area.

Guide wire 1411 is generally inserted first and guided to the area of the diseased or faulty valve. The catheter 1401 is advanced along the guide wire 1411 until the compressed valve 900′ is at the diseased valve. In some procedures, the compressed valve 900′ may be inserted directly inside the diseased valve.

In one embodiment of the invention, the compressed valve 900′ is removably collapsed around a deflated balloon 1402′ as shown in FIG. 14B. The balloon 1402′ is temporarily located within both the open leaflets and the frame/stent. A pump or syringe 1410 of the catheter 1400 may then be used to pump a fluid such as saline down the catheter shaft to expand the deflated balloon 1402′ into its expanded state of the balloon 1402. The catheter 1401 may include an optional removable sheath 1431 that may be slid over the deflated balloon 1402′ and the compressed valve 900′for insertion. For delivery, the optional sheath 1431 is retracted such as illustrated in FIG. 14C.

In FIG. 14C, the distal end of the catheter with the compressed valve 900′ collapsed around the deflated balloon 1402′ is advanced inside a vessel 1460. When the compressed valve 900′ reaches the desired location inside the vessel 1460, the deflated balloon 1402′ is inflated to expand the compressed valve 900′ into the non-compressed or expanded valve 900.

FIG. 14D illustrates the inflated balloon 1402 with expanded valve 900 inside vessel 1460. Deflated balloon 1402′ may be inflated using inflation syringe 1410 filled with a saline solution. The inflation syringe 1410 is coupled to an inlet 1420 and used to inject saline solution through the stopcock 1422. The saline solution travels through catheter 1401 and inflates the deflated balloon 1402′ thereby expanding valve 900′ to expanded valve 900. The expanded valve 900 may be expanded to a diameter slightly greater than the diameter of the vessel 1460 such that the sides of the valve 900 anchor to the sidewalls of vessel 1460. Stopcock 1422 may be closed to maintain pressure in the inflated balloon 1402. Once the valve 900 is deployed, stopcock 1422 is opened to relieve the fluid pressure inside the catheter and deflate the balloon 1402. The catheter 1401 and balloon 1402′ are then removed leaving the valve 900 behind.

A catheter delivery system may also be used to deliver a compressed self-expanding valve. In one embodiment, the compressed self-expanding valve would be placed within restrictive structure of the delivery system, to prevent the expansion of the valve, prior to placement within a body. The frame or stent of the compressed valve may be constructed of a shape memory metal or polymer such as Nitinol. The valve would be pushed out of the restrictive structure or the restrictive structure could be pulled away from the valve and the valve allowed to self-expand to its original shape within the vessel.

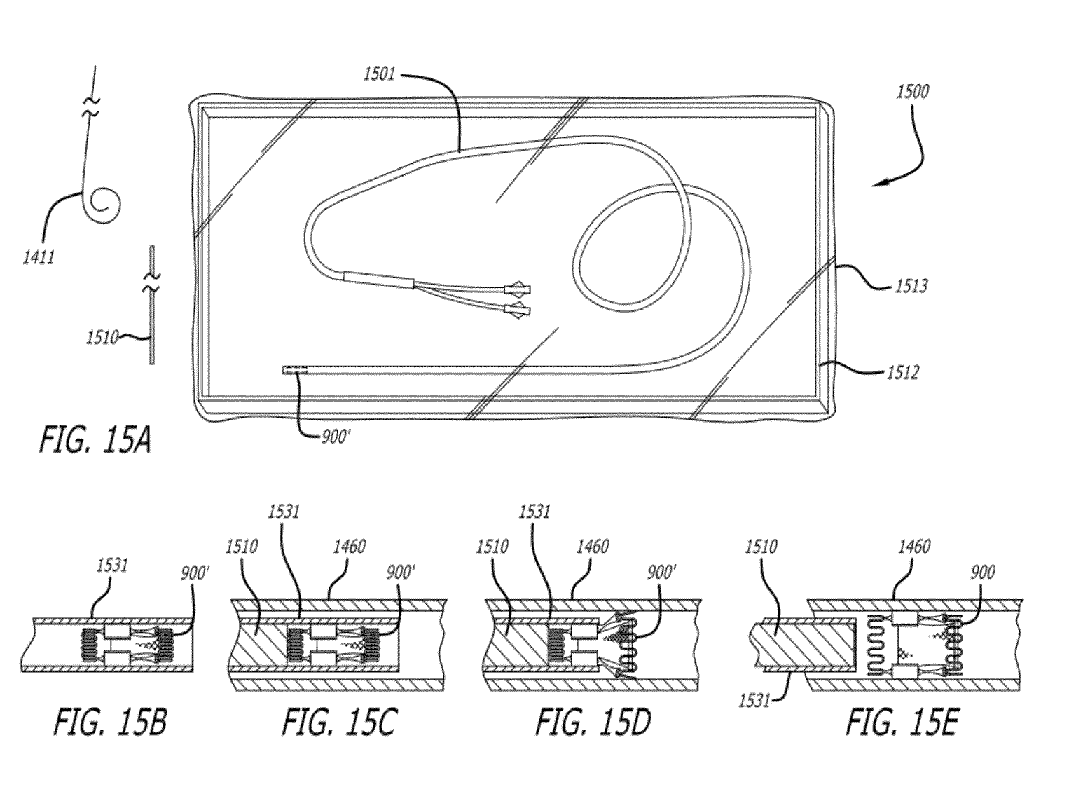

Referring now to FIG. 15A, a catheter delivery kit and system 1500 for a self-expanding prosthetic valve is shown. System 1500 comprises catheter 1501 with a compressed valve 900″ removably coupled thereto inside a hermetically sealed sterile container 1513. The container 1513 provides a sterile barrier to maintain the contents, including the catheter 1501 and the valve 900′, in a sterile condition during shipping and storage, until ready for surgery. A pushrod 1510 and guide wire 1411 may be included as part of the kit 1500 and optionally contained within the container 1513. The compressed self-expanding valve 900′ is located at the distal end of the catheter, ready for insertion and deployment.

In FIG. 15B, the compressed self-expanding valve 900′ is shown inside catheter 1501. The tube 1531 of the distal tip of catheter 1501 acts as a restrictive structure to mechanically restrain the shape memory stent of the compressed self-expanding valve 900′ from self-expanding prior to proper position in the body. The tubular catheter 1501 is advanced inside vessel 1460 until the valve 900′ is in a proper position to be deployed. Pushrod 1510 may act as a stop to prevent the valve 900′ from sliding backwards into the tube 1531 of the catheter 1501 during advancement of the catheter.

In FIG. 15C, the compressed self-expanding valve 900′ has reached a proper deployment position and a pushrod 1510 inserted into the tube 1531 is shown ejecting the valve 900′ out of an open end from the restrictive tubular structure 1531 of catheter 1501. Once free of the confining tip of catheter 1501, the valve 900′ expands to its uncompressed state, securing the valve to the sidewalls of the vessel 1460. A shape memory stent that is a self expanding stent may be preferred for use in a self-expanding valve. A shape memory stent will warm up from the body heat of the patient and return to its uncompressed state with little to no deformation. Once the compressed valve 900′ has been deployed as the uncompressed valve 900 and secured to the vessel 1460, the catheter may be withdrawn. The size (e.g., diameter) of the uncompressed valve and stent is selected to match the vessel or artery in which it is to be deployed. Uncompressed prosthetic valves are typically provided in diameters from ten millimeters (10 mm) to thirty-five millimeters (35 mm), inclusive, in either odd or even size designations.

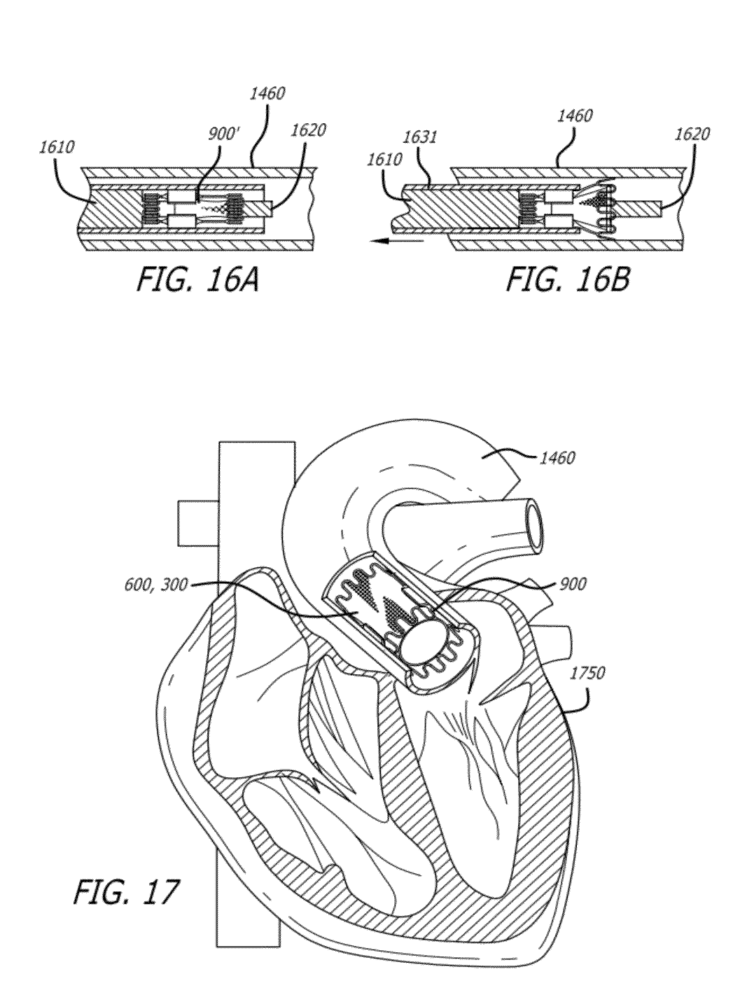

Referring now to FIG. 16A-16B, in an alternate embodiment of the delivery kit 1500 for the compressed self expanding prosthetic valve 900′, an anchor tab 1620 extends from a rod 1610 out of the catheter. The anchor tab 1620 may be a hook, gripper, clip, or an inflatable balloon. The anchor tab 1620 may be used to anchor to the sidewall of the vessel 1460 to aid in ejecting the compressed self expanding prosthetic valve 900′ from the tube 1631 near the tip of catheter. Instead of using a pushrod 1510 to fully eject the compressed valve 900′, the anchor tab 1620 may be secured to the vessel 1460 and the compressed valve 900′may be pulled out of the restrictive structure by withdrawing the tube 1631 of the catheter. It may be advantageous to pull instead of push the compressed valve 900′ out of the catheter to reduce deformation of the stent and damage to the valve 900. Once the valve 900 is deployed, the anchor tab 1620 may be released and fully withdrawn with the catheter.

Referring now to FIG. 17, an uncompressed prosthetic valve 900,600,300 is shown in position within a heart 1750. In particular, the valve 900,600,300 is inserted within a vessel 1460, such as the aorta. Once inserted, the prosthetic valve 900.600,300 will passively open and close to control the flow of oxygenated blood through the left side of the heart and into the aorta for distribution to the body. Specifically, when the pressure in the left ventricle rises above the

Doc. No. 2010.P001US pressure in the aorta, the valve 900,600,300 opens, allowing blood to exit the left ventricle into the aorta.

### Conclusion

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. All of these embodiments are intended to be within the scope of the invention herein disclosed. These and other embodiments of the present invention will become readily apparent to those skilled in the art after reading the detailed description of the embodiments of the invention having reference to the attached figures, the invention not being limited to any particular preferred embodiment(s) disclosed since various other modifications may occur to those ordinarily skilled in the art. Accordingly, the claimed invention is limited only by claims that follow below.

1-80. (canceled)

81. A kit for implanting a prosthetic valve into a vessel in the body of a mammal, the kit comprising:
a container which provides a sterile barrier;
a sterile catheter within the container, the sterile catheter having a distal end and a proximal end; and
a sterile collapsed prosthetic valve within the container removably coupled to the distal end of the sterile catheter, wherein the sterile collapsed prosthetic valve includes one or more prosthetic valve leaflets formed from an isotropic screen material.

82. The kit of claim 81, wherein
the isotropic screen material has openings with a pore size inclusively between fifteen microns and sixty microns, and a thickness inclusively between one-hundredth of a millimeter and one-tenth of a millimeter.

83. The kit of claim 81, wherein
the sterile collapsed prosthetic valve further includes
two or more prosthetic valve leaflets, and
a support structure coupled to the two or more prosthetic valve leaflets to support them between commis sure portions.

84. The kit of claim 83, wherein
the support structure of the sterile collapsed prosthetic valve is self expanding, and
the sterile collapsed prosthetic valve is constrained in a collapsed state awaiting release from the catheter for self expansion into a vessel.

85. The kit of claim 84, further comprising:
a push rod inserted into the proximal end of the catheter, the push rod to push out on the sterile collapsed prosthetic valve to release it from the catheter and allow the valve to self expand into the vessel

86. The kit of claim 84, further comprising:
a pull rod inserted into the proximal end of the catheter, the pull rod to pull out on the sterile collapsed prosthetic valve to release it from the catheter and allow the valve to self expand into the vessel

87. The kit of claim 84, further comprising:
a guide wire insertable into the proximal end of the catheter, the guide wire to guide the distal end of the catheter to a position within the vessel to release the sterile collapsed prosthetic valve from the catheter for self expansion into the vessel.

88. The kit of claim 83, wherein
the support structure of the sterile collapsed prosthetic valve is in a collapsed state and expandable,
the catheter includes an expansion device at the distal end, and
the sterile collapsed prosthetic valve is mounted over the expansion device awaiting release from the catheter and expansion into a vessel by the expansion device.

89. The kit of claim 88, wherein
the expansion device is an expandable balloon responsive to fluid pressure, the expandable balloon to expand in response to increased fluid pressure and to contract in response to reduced fluid pressure.

90. The kit of claim 89, further comprising:
a syringe to couple to the catheter near the proximal end to provide the fluid pressure to expand and contract the expandable balloon.

91. The kit of claim 90, further comprising:
a guide wire insertable into the proximal end of the catheter to guide the distal end of the catheter to a position within the vessel to expand the sterile collapsed prosthetic valve out into the vessel.

92. The kit of claim 90, wherein
the catheter further includes a stopcock valve, the stopcock valve to close and hold the fluid pressure to expand the expandable balloon and the sterile collapsed prosthetic valve out into the vessel, and the stopcock valve to open to allow the fluid pressure to be reduced, contracting the expandable balloon and releasing the catheter from the prosthetic valve.

93. The kit of claim 81, wherein
the prosthetic valve leaflet is formed with one or more sealed edges so that it can be handled without coming apart.

94. The kit of claim 93, wherein
the isotropic screen material is a monofilament polyester or polypropylene to form an isotropic structure with uniform pore size.

95. The kit of claim 93, wherein
the shape of the prosthetic valve leaflet is cut from the isotropic screen material using a laser or other non-contact method to form the one or more sealed edges.

96. The kit of claim 81, wherein
the container further provides support and protects the catheter from damage.

97. The kit of claim 96, wherein
the container is a tray with a lid, or wrap.

98. The kit of claim 96, wherein
the container is a tray with a plastic wrap.

99. The kit of claim 81, wherein
the container is a bag.

100. A method for a kit to implant a prosthetic valve into a vessel, the method comprising:
providing a collapsed prosthetic valve including one or more prosthetic valve leaflets formed from an isotropic screen material;
removably coupling the collapsed prosthetic valve to a catheter;
sealing the collapsed prosthetic valve and the catheter into a container; and
sterilizing the collapsed prosthetic valve and the catheter for later surgical use.

101. The method of claim 100, further comprising:
prior to sealing, setting the collapsed prosthetic valve and the catheter onto a tray and further sealing the tray within the container to support and protect the collapsed prosthetic valve and the catheter from damage before, during and after sterilization and during storage.

102. The method of claim 100, further comprising:
opening the sealed container;
inserting the sterile catheter with the sterile collapsed prosthetic valve removeably coupled thereto into a vessel;
guiding a distal end of the sterile catheter with the sterile collapsed prosthetic valve through the vessel into a valve position;
expanding the sterile collapsed prosthetic valve into the vessel; and
withdrawing the catheter from the vessel.

103. The method of claim 102, wherein the sterile collapsed prosthetic valve includes a self expanding support structure and the expanding of sterile collapsed prosthetic valve into the vessel comprises
pushing the sterile collapsed prosthetic valve into the vessel such that the self expanding support structure expands the valve into the vessel.

104. The method of claim 102, wherein the sterile collapsed prosthetic valve includes a self expanding support structure and the expanding of sterile collapsed prosthetic valve into the vessel comprises
pulling on the catheter to release the sterile collapsed prosthetic valve into the vessel such that the self expanding support structure expands the valve into the vessel.

105. The method of claim 102, wherein the expanding of sterile collapsed prosthetic valve into the vessel comprises
expanding a balloon of the catheter to push out on a support structure of the sterile collapsed prosthetic valve into the vessel.
